(19) United States
(12) Patent Application Publication
(10) Pub. No.: US 2008/0188714 A1
(43) Pub. Date: Aug. 7, 2008

(54) ELECTROMECHANICAL IN-SITU CLEANING OF OPTICAL ELEMENTS

(75) Inventor: Nathaniel J. McCaffrey, Hampton Falls, NH (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: BOSTON SCIENTIFIC SCIMED, INC., Maple Grove, MN (US)

(21) Appl. No.: 11/672,476

(22) Filed: Feb. 7, 2007

(51) Int. Cl. A61B 1/012

(52) U.S. Cl. 600/157

(57) ABSTRACT

A medical device for imaging internal body cavities of a patient includes one or more optical components and one or more vibrating elements. The vibrating elements are selectively activated during the application of a washing liquid over the optical components to enhance the cleaning ability of the liquid.

### FIELD OF THE INVENTION

The present invention relates to medical devices in general, and, in particular, to medical devices with optical elements for visualizing internal body cavities of patients.

### BACKGROUND OF THE INVENTION

As an alternative to more invasive surgical procedures, it is becoming increasingly common for physicians to use endoscopes or other medical imaging devices to view internal body cavities of patients. However, during use, the optical elements of such devices that provide images of the patient often become obscured by blood, mucus, or other matter encountered in the patient's body. To clear the optical components, many devices include a lens-washing feature that directs a washing liquid, typically water or saline, and/or a gas, such as air or carbon dioxide, across the surface of the optics. While the application of a washing liquid or gas across the optical components has proven satisfactory in the past in order to improve image quality, such techniques may not perform adequately when used with high resolution image sensors and other optical elements. In addition, some optical components, such as plastic lenses, do not clean as well as glass components when subjected to a stream of washing liquid alone. Therefore, such components have not been widely incorporated into such medical devices, despite their lower cost.

### SUMMARY OF THE INVENTION

To address the problems described above, the present invention is a medical imaging device including one or more optical components, such as windows or lenses and one or more vibrating elements, such as ultrasonic transducers. The one or more vibrating elements are selectively energized to improve the cleaning action of a lens-washing liquid that is applied to the optical components.

In one specific embodiment, the medical device is an endoscope that is equipped with one or more ultrasonic transducers positioned in the distal tip. The ultrasonic transducers are energized during application of a washing liquid towards the optical components in order to enhance the cleaning action of the washing liquid. The ultrasonic transducers may be powered by an ultrasonic generator which is either in the endoscope itself, or in a separate control unit. The ultrasonic transducers may be activated by a physician using a lens wash or other button on the endoscope. Alternatively, an image processor can be used to determine when the optical components need to be cleaned and the image processor activates the ultrasonic transducers during cleaning.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 illustrates a medical device with one or more vibrating elements, such as ultrasonic transducers, in accordance with one embodiment of the present invention; and

FIG. 2 is a block diagram of a medical device including vibrating elements in accordance with an embodiment of the present invention.

### DETAILED DESCRIPTION OF THE PREFERRED EMBODIMENT

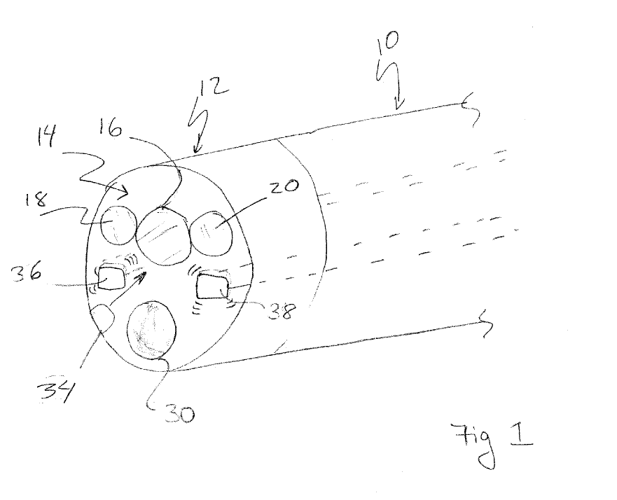

The present invention is a medical device including one or more vibrating elements for cleaning optical components of the device. As shown in FIG. 1, a medical device 10 comprises an endoscope or other imaging device that can be inserted into an internal body cavity of a patient. A representative endoscope suitable for use with the present invention is disclosed in U.S. patent application Ser. No. 10/956,007, Publication No. US2005-0197536 A1, filed Sep. 30, 2005, which is herein incorporated by reference. The endoscope 10 includes a distal tip 12 with a number of optical components 14 that produce images of the patient's tissues. The optical components 14 generally include a window or front element of a lens assembly 16 that is positioned in front of an image sensor (not shown) or in front of a fiber optic imaging guide that transfers an image to the proximal end of the device. In addition, the optical components include one or more windows or lenses 18, 20 that are positioned in front of illumination sources. Such illumination sources may include light-emitting diodes, fiber optic illumination guides, or the like. Typically, the windows or lenses 18, 20 in front of the illumination sources are positioned near the window or the lens assembly 16 in order to produce evenly illuminated images of the tissue that is in the area of the distal tip 12.

The medical device 10 also includes one or more working channels 30 that allow the introduction of additional medical devices into the patient to perform such tasks as obtaining biopsy samples and/or performing surgical procedures.

To clean the optical components, including the window or lens assembly 16 and/or the lenses or windows 18, 20 that are in front of the illumination sources, the endoscope includes a lens wash nozzle 34 that directs a stream of washing liquid and/or a gas across the surface of the optical components.

To enhance the ability of the lens-washing liquid to clean the optical components 14, the present invention includes one or more vibrating elements, such as ultrasonic transducers 36, 38, that are positioned to vibrate the lens washing liquid, the optical components 14, or both during cleaning. Vibration of the ultrasonic transducers 36, 38 during the application of a lens wash liquid toward the optical components may cause cavitation or bubbles in the lens wash liquid to improve its cleaning ability. The ultrasonic transducers 36, 38 may be positioned on the distal face or exterior of the endoscope. Alternatively, the ultrasonic transducers 36, 38 may be positioned on the interior of the endoscope such that, upon activation, the transducers vibrate the distal tip to improve the cleaning ability of the lens wash liquid.

Although the ultrasonic transducers 36, 38 are shown in FIG. 1 as being generally rectangular in shape, it will be appreciated that other shapes of transducers could be used. In one embodiment, the transducers may comprise ring-like structures which surround one or more of the optical components or they may be contoured to fit in the free space between the various optical components at the distal tip. The ultrasonic transducers 36, 38 may be formed from piezoelectric crystals that are activated to vibrate upon application of an electronic driving signal from an ultrasound generator as described below. However, other types of vibrational transducers, such as motors similar to the type found in cellular phone ringers could also be used to impart ultrasonic vibrations in the washing liquid or the optical components. In yet another embodiment, an optically clear material that also electrically conductive may be placed directly in front of, or bonded to, the optical components and used as an ultrasonic vibrator. In some embodiments, the optical components themselves may be made of the transparent, electrically conductive material. One suitable optically transparent and conductive material may be SnO2 that can be used as a piezoelectric device.

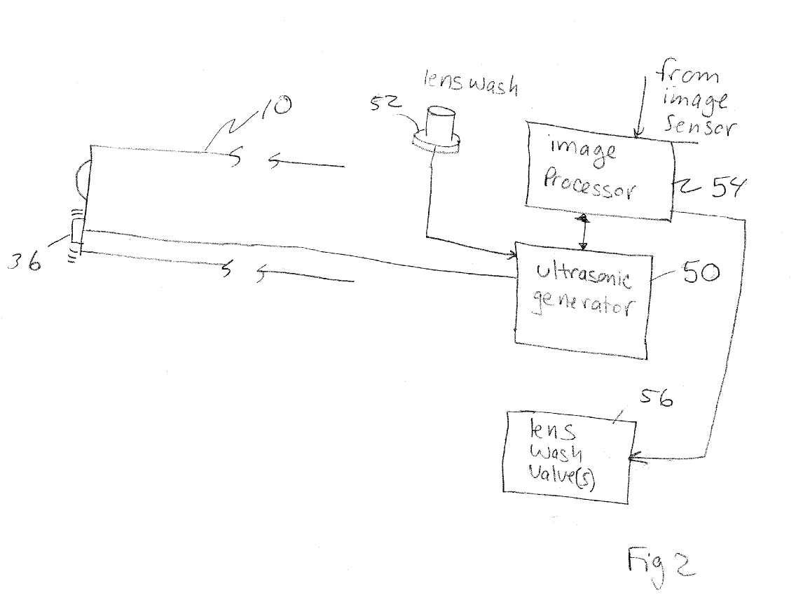

FIG. 2 illustrates an ultrasonic generator 50 that provides driving signals to one or both of the ultrasonic transducers 36, 38 at the distal end of the endoscope 10 in accordance with one embodiment of the present invention. The ultrasonic generator 50 may be activated by a lens wash button 52 that is located on the endoscope itself, such as on a handle. However, the lens wash button may be located in other locations, such as on a foot switch or on a remote console, for activation by a physician or their assistant.

In another alternative embodiment, the ultrasonic generator 50 is activated by an image processor 54 that receives signals from an image sensor (not shown). The image processor 54 analyzes images of the patient's tissue to determine if the optical components are occluded or otherwise need to be cleaned. The image processor may analyze the images to determine if a cleaning cycle is required by searching for areas of reduced brightness or low contrast pixels that are present in successive images despite different orientations of the distal tip of the device.

Upon determination that a cleaning cycle is necessary, the image processor 54 activates the ultrasonic generator 50 and one or more lens wash valves 56 that supply the washing liquid across the optical components of the endoscope.

Although the present invention is described with respect to endoscopes such as colonoscopes, duodenoscopes, bronchoscopes, and the like, it will be appreciated that the invention could be used in any medical device having components that need to be cleaned periodically while in the patient. The application of the vibrational and, particularly, ultrasonic energy during cleaning enhances the ability of an applied cleaning solution to clean the components.

While the preferred embodiment of the invention has been illustrated and described, it will be appreciated that various changes can be made therein without departing from the scope of the invention. For examples, in some instances, activation of the vibrational transducers alone may be sufficient to clean the optical components at the distal tip without application of a washing liquid. It is, therefore, intended that the scope of the invention be determined from the following claims and equivalents thereof.

The embodiments of the invention in which an exclusive property or privilege is claimed are defined as follows:

1. A medical device, comprising:
a shaft having a proximal end and a distal end;
one or more optical components at the distal end of the shaft;
a port that directs a washing liquid over the optical components; and
one or more vibrational transducers that are positioned near the optical components and are selectively enabled to vibrate as a washing liquid is directed over the optical components.

2. The medical device of claim 1, wherein the one or more optical components includes a lens at the distal tip of the shaft and the one or more vibrational transducers are ultrasonic transducers that are positioned adjacent to the lens.

3. The medical device of claim 1, wherein the one or more optical components includes a window at the distal tip of the shaft and the one or more vibrational transducers are ultrasonic transducers that are positioned adjacent to the window.

4. The medical device of claim 1, further comprising an ultrasonic generator for producing driving signals to the one or more vibrational transducers.

5. The medical device of claim 4, wherein the medical device further includes a handle, and the ultrasonic generator is in the handle.

6. The medical device of claim 4, wherein the medical device is connectable to a control cabinet, and the ultrasonic generator is in the control cabinet.

7. The medical device of claim 4, wherein the ultrasonic generator is in the shaft.

8. The medical device of claim 4, wherein the medical device includes a lens wash button that activates the delivery of a washing liquid towards the optical components, and, wherein, the ultrasonic generator is activated by the lens wash button.

9. The medical device of claim 4, wherein the delivery of a washing liquid towards the optical components is controlled by a processor that analyzes images of tissue to determine when the optical components need to be cleaned and activates the ultrasonic generator during delivery of the washing liquid.

10. A medical device, comprising:
a shaft having a proximal end and a distal end;
one or more optical components at the distal end of the shaft; and
one or more vibrational transducers that are positioned near the optical components and are selectively enabled to vibrate in order to clean the optical components.

11. The medical device of claim 10, wherein the one or more vibrational transducers are piezoelectric transducers that are energized to clean the optical components.

12. The medical device of claim 10, wherein the one or more vibrational transducers include a vibrating electric motor.

13. The medical device of claim 10, wherein the one or more vibrational transducers include an optically transparent, electrically conductive material that functions as a piezoelectric transducer when a driving signal is applied.
